(19) United States
(12) Patent Application Publication
(10) Pub. No.: US 2016/0120505 A1
(43) Pub. Date: May 5, 2016

(54) ULTRASONIC PROBE

(71) Applicant: Konica Minolta Inc., Tokyo (JP)

(72) Inventor: Masahiko KADOKURA, Sagamihara-shi (JP)

(21) Appl. No.: 14/928,538

(22) Filed: Oct. 30, 2015

(30) Foreign Application Priority Data
Nov. 4, 2014 (JP) 2014-224294

(51) Int. Cl. A61B 8/00; A61B 8/12

(52) U.S. Cl. CPC A61B 8/4461; A61B 8/12; A61B 8/4494; A61B 8/4281

(57) ABSTRACT

An ultrasonic probe includes: a transducer unit including a plurality of transducers transmitting/receiving an ultrasonic wave; a transducer holding frame; an acoustic window formed from a material having an ultrasonic transmission property, the acoustic window forming a first space including the transducer unit in an inner part; a driving unit; a driving unit holding frame which holds the driving unit and forms a second space including an output shaft of the driving unit in an inner part; a pipe including a hollow portion with which connects the first space and the second space; a force transmission unit; and a housing including, in an inner part, the transducer unit, the transducer holding frame, the acoustic window, the driving unit, the driving unit holding frame, the pipe, and the force transmission unit, wherein the first space, the hollow portion of the pipe, and the second space are filled with coupling liquid.

The entire disclosure of Japanese Patent Application No. 2014-224294 filed on Nov. 4, 2014 including description, claims, drawings, and abstract are incorporated herein by reference in its entirety.

### BACKGROUND OF THE INVENTION

1. Field of the Invention

The present invention relates to an ultrasonic probe which is inserted into a body cavity and which performs scanning with an ultrasonic transducer automatically.

2. Description of the Related Art

Recently, an ultrasonic diagnostic device which includes an ultrasonic probe and which images the echo signal has been widely used, the ultrasonic probe being inserted into a body cavity such as a rectum or a vagina and acquiring an echo signal which is a reflection wave of an ultrasonic wave transmitted into the body cavity.

As an ultrasonic probe which is inserted into a body cavity in such a manner and which performs scanning with an ultrasonic transducer automatically, what performs scanning with an ultrasonic wave by making a transducer unit, which includes a plurality of transducers to transmit/receive an ultrasonic wave and which is arranged at a leading end part, perform an oscillating movement by drive force (JP 10-179588 A and JP 2000-70268 A) is generally used.

It is desired to further improve operability of an ultrasonic probe for a body cavity.

### SUMMARY OF THE INVENTION

The present invention has been made in view of the forgoing and an object thereof is to provide an ultrasonic probe for a body cavity with which probe operability is improved, for example, by reducing weight.

To achieve the abovementioned object, according to an aspect, an ultrasonic probe reflecting one aspect of the present invention comprises: a transducer unit including a plurality of transducers configured to transmit/receive an ultrasonic wave; a transducer holding frame which holds the transducer unit in an oscillatable manner; an acoustic window which is formed from a material having an ultrasonic transmission property, provided in a front part of an ultrasonic wave transmission/reception surface of the transducer unit, and fixed to the transducer holding frame in a sealed manner, the acoustic window forming a first space including the transducer unit in an inner part; a driving unit configured to generate drive force to oscillate the transducer unit; a driving unit holding frame which holds the driving unit and forms a second space including an output shaft of the driving unit in an inner part; a pipe including a hollow portion with which connects the first space and the second space; a force transmission unit which is put through the hollow portion of the pipe and which is configured to oscillate the transducer unit by transmitting the drive force of the driving unit; and a housing including, in an inner part, the transducer unit, the transducer holding frame, the acoustic window, the driving unit, the driving unit holding frame, the pipe, and the force transmission unit, wherein the first space, the hollow portion of the pipe, and the second space are filled with coupling liquid.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is an upper sectional view of an ultrasonic probe according to an embodiment of the present invention;

FIG. 2 is a front sectional view of the ultrasonic probe according to the embodiment of the present invention;

FIG. 3 is a partial perspective view of the ultrasonic probe according to the embodiment of the present invention;

FIG. 4A and FIG. 4B are views for describing a method of fixing a wire;

FIG. 5 is a partial perspective view of the ultrasonic probe according to the embodiment of the present invention; and

FIG. 6 is a partial perspective view of the ultrasonic probe according to the embodiment of the present invention.

### DESCRIPTION OF THE PREFERRED EMBODIMENTS

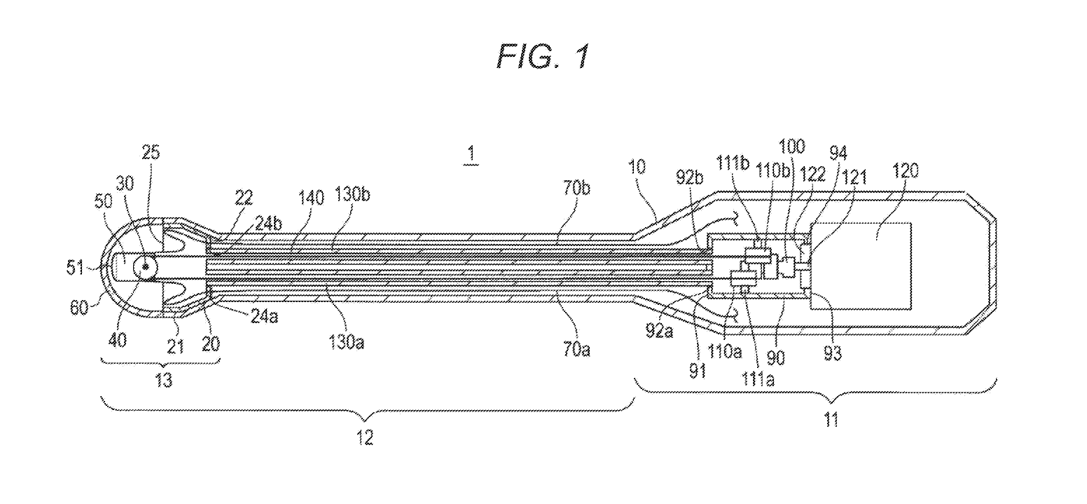

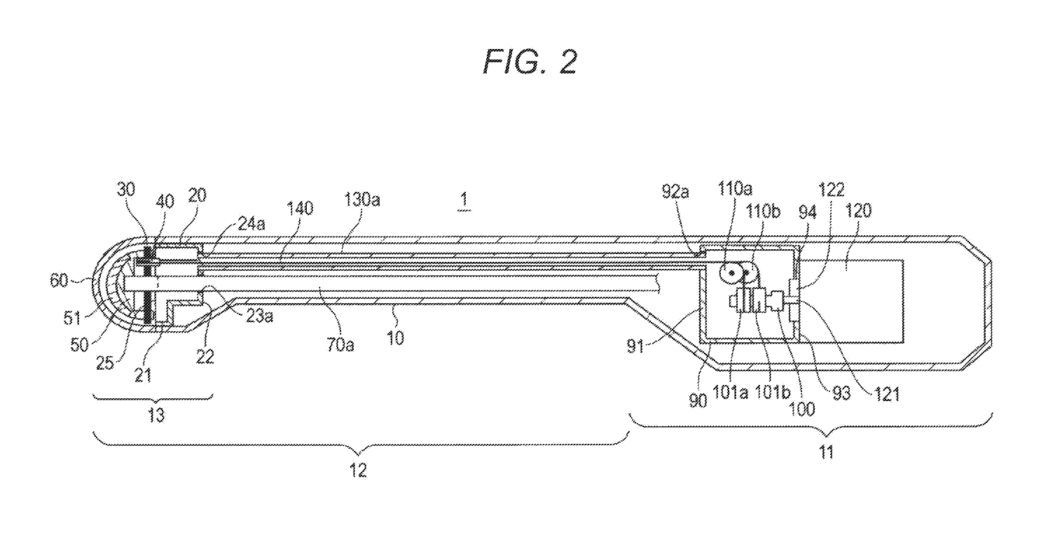

Hereinafter, an embodiment of the present invention will be described with reference to the drawings. However, the scope of the invention is not limited to the illustrated examples. FIG. 1 and FIG. 2 are respectively an upper sectional view and a front perspective view of an ultrasonic probe according to an embodiment of the present invention. As illustrated in FIG. 1 and FIG. 2, a housing 10 of an ultrasonic probe 1 includes a grip part 11 gripped by an operator in the outside of a body cavity and an insertion part 12 inserted into the body cavity.

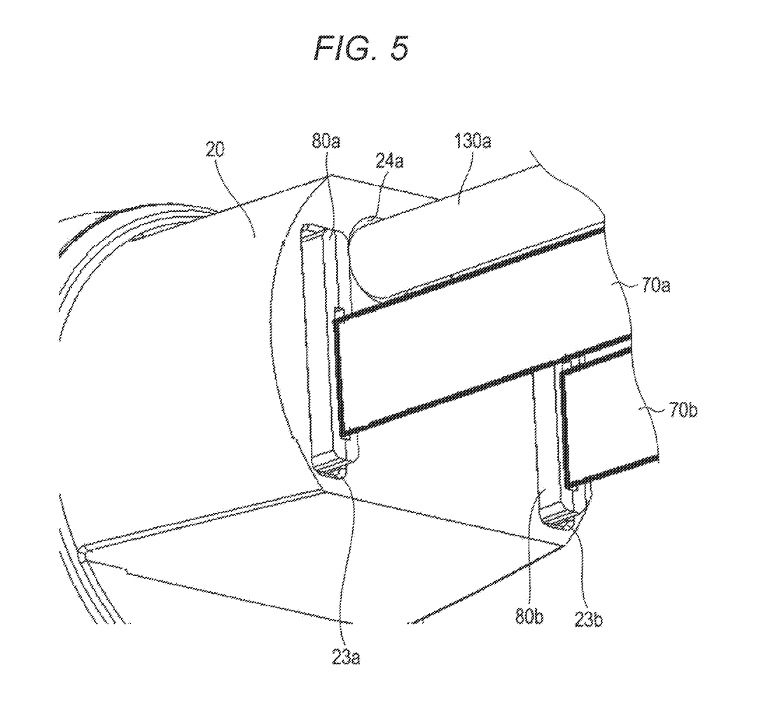

In an inner part of a leading end part 13 of the insertion part 12, a transducer holding frame 20 is attached in a fixed manner. The transducer holding frame 20 is formed from a material such as aluminum or plastic in a hollow shape with an opening at one end. In an outer peripheral part of a side surface 21, the transducer holding frame 20 is fixed to an inner periphery of an end part of the insertion part 12 of the housing 10. On a bottom surface 22 of the transducer holding frame 20, holes 23a and 23b (see FIG. 5) through which flexible printed circuits (FPC) 70a and 70b are put and holes 24a and 24b to which pipes 130a and 130b are attached are provided.

To the transducer holding frame 20, an oscillation shaft 30 is attached in a rotatable manner. The oscillation shaft 30 is formed in a cylindrical shape from a material such as stainless steel. To the oscillation shaft 30, a driven pulley 40 and a transducer unit 50 are attached in a fixed manner.

The driven pulley 40 oscillates the oscillation shaft 30 along with a movement of a wire (force transmission unit) 140 due to driving of a motor (driving unit) 120. Note that a detail of a method of arranging the wire 140 to the driven pulley 40 will be described later.

To the transducer unit 50, a plurality of transducers (piezoelectric oscillation element) to transmit/receive an ultrasonic wave is arranged. Each transducer converts an electric signal into an ultrasonic wave and emits the ultrasonic wave to an inner side of a body of a subject. Then, each transducer detects the ultrasonic wave reflected in the inner side of the body (reflection wave) and converts the ultrasonic wave into an electric signal. Along with oscillation of the oscillation shaft 30, the transducer unit 50 oscillates around a central axis of the oscillation shaft 30 extended in a direction orthogonal to an ultrasonic wave transmission/reception surface 51 (up/down direction on plane of paper in FIG. 2). By oscillating the transducer unit 50, it is possible to move an ultrasonic wave forming surface and to form a three-dimensional ultrasonic image.

In a front part of the ultrasonic wave transmission/reception surface 51 of the transducer unit 50, an acoustic window 60 is attached. The acoustic window 60 is formed in a dome shape from a material having an ultrasonic transmission property and is fixed to the transducer holding frame 20 in a sealed manner to cover an opening 25 of the transducer holding frame 20. As a result, the transducer unit 50 is arranged in a space formed by the acoustic window 60 and the transducer holding frame 20 (first space).

The space formed by the acoustic window 60 and the transducer holding frame 20 (first space) is filled with coupling liquid to improve propagation capability of an ultrasonic wave and to guide the ultrasonic wave to a body surface with a little attenuation.

To each transducer arranged in the transducer unit 50, the flexible printed circuits 70a and 70b are connected. The flexible printed circuits 70a and 70b are pulled from the holes 23a and 23b provided in the bottom surface 22 of the transducer holding frame 20. Each of the flexible printed circuits 70a and 70b transmits, to each transducer, an electric signal output from a diagnosis device main body (not illustrated) and transmits, to the diagnosis device main body, an electric signal output from each transducer. Note that a detail of a method of sealing the holes 23a and 23b will be described later.

Further, into the holes 24a and 24b provided in the bottom surface 22 of the transducer holding frame 20, one end parts of the pipes 130a and 130b are respectively inserted. In this state, the pipes 130a and 130b are welded to the transducer holding frame 20 and the holes 24a and 24b are sealed. Note that the space formed by the acoustic window 60 and the transducer holding frame 20 are in communication with the hollow portions of the pipes 130a and 130b. Thus, the hollow portions of the pipes 130a and 130b are also filled with the coupling liquid.

In an inner part of the grip part 11 of the housing 10, a casing (driving unit holding frame) 90 is attached in a fixed manner. The casing 90 is formed, for example, in a hollow shape from a metal or plastic material. In a first surface 91 of the casing 90 which surface faces the transducer holding frame 20, holes 92a and 92b to which the pipes 130a and 130b are attached are provided. In a second surface 93 of the casing 90 which surface faces the transducer holding frame 20, a hole 94 through which an output shaft 121 of the motor 120 is inserted into an inner part is provided.

To the second surface 93 of the casing 90, the motor 120 is attached in a fixed manner in a state in which the output shaft 121 of the motor 120 is inserted into the hole 94. The motor 120 functions as a driving mechanism to oscillate the transducer unit 50. An oil seal 122 is fitted between the hole 94 and the output shaft 121 of the motor 120. Accordingly, the hole 94 is sealed.

Further, into the holes 92a and 92b provided in the first surface 91 of the casing 90, the other end parts of the pipes 130a and 130b are respectively inserted. In this state, the pipes 130a and 130b are welded to the casing 90 and the holes 92a and 92b are sealed. Note that an inner space of the casing 90 is in communication with the hollow portions of the pipes 130a and 130b. Accordingly, the inner space of the casing 90 is also filled with the coupling liquid.

In the inner space of the casing 90 (second space), a driving pulley unit 100 and intermediate pulleys 110a and 110b are housed.

The driving pulley unit 100 is coaxially attached to a leasing end of the output shaft 121 of the motor 120 in a fixed manner. In the driving pulley unit 100, driving pulleys 101a and 101b are formed. A rotational axis each of the driving pulleys 101a and 101b is orthogonal to a rotational axis of the driven pulley 40. Note that a detail of the driving pulley unit 100 will be described later.

The intermediate pulleys 110a and 110b are attached to the casing 90, respectively through the attachment shafts 111a and 111b, in a manner rotatable around the attachment shafts 111a and 111b or rotatable along with the attachment shafts 111a and 111b. A rotational axis of each of the intermediate pulleys 110a and 110b is orthogonal to the rotational axis of the driven pulley 40 and the rotational axis of each of the driving pulleys 101a and 101b. In an outer periphery of each of the intermediate pulleys 110a and 110b, a wire groove to arrange the wire 140 is formed.

The pipes 130a and 130b are formed from a metal material. One end parts of the pipes 130a and 130b are respectively inserted into the holes 24a and 24b and fixed to the transducer holding frame 20 and the other end parts thereof are respectively inserted into the holes 92a and 92b and fixed to the casing 90. The hollow portions of the pipes 130a and 130b are in communication with the space formed by the acoustic window 60 and the transducer holding frame 20 and the inner space of the casing 90. Thus, the hollow portions of the pipes 130a and 130b and the inner space of the casing 90 are also filled with the coupling liquid.

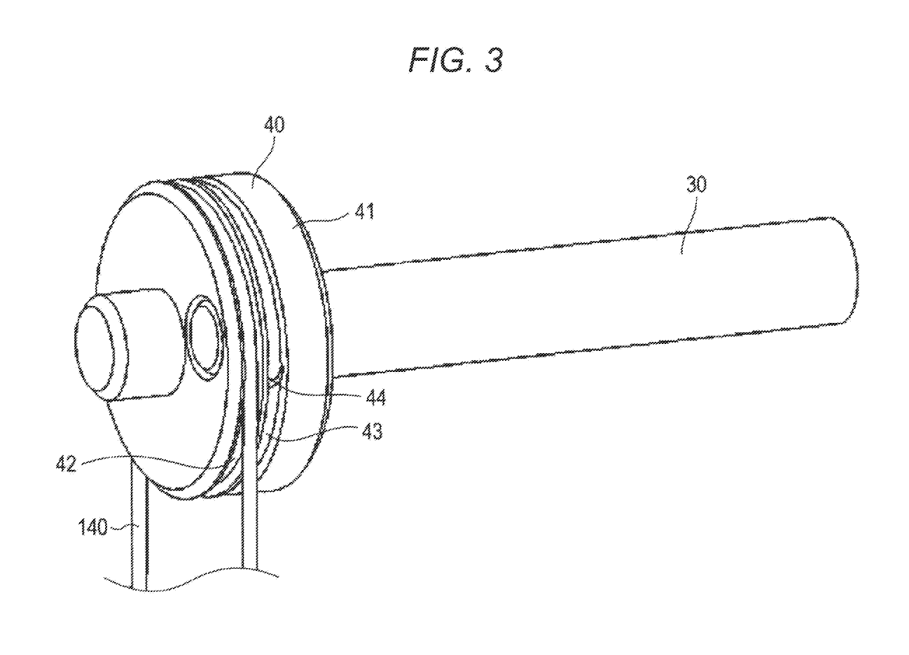

The wire 140 is serially arranged in the driving pulley 101a and the intermediate pulley 110a while one end part thereof is fixed to the driving pulley 101a. Then, the wire 140 is put through the pipe 130a, arranged in the driven pulley 40, and put through the hole 44 (see FIG. 3) of the driven pulley 40. Further, the wire 140 put though the hole 44 is serially arranged in the driven pulley 40, put through the pipe 130b, and arranged in the intermediate pulley 110a and the driving pulley 101b. Then, the other end part of the wire 140 is fixed to the driving pulley 101b.

Then, a detail of a method of arranging the wire 140 to the driven pulley 40 will be described with reference to FIG. 3. As illustrated in FIG. 3, in an outer peripheral surface 41 of the driven pulley 40, two wire grooves 42 and 43 are provided and the hole 44 to connect the wire groove 42 and the wire groove 43 is formed. To the driven pulley 40, the wire 140 arranged in the wire groove 42 is arranged doubly in such a manner that the wire 140 is also arranged in the wire groove 43 through the hole 44. The wire 140 is fixed to the driven pulley 40, in a part of the hole 44, with a pin member or the like.

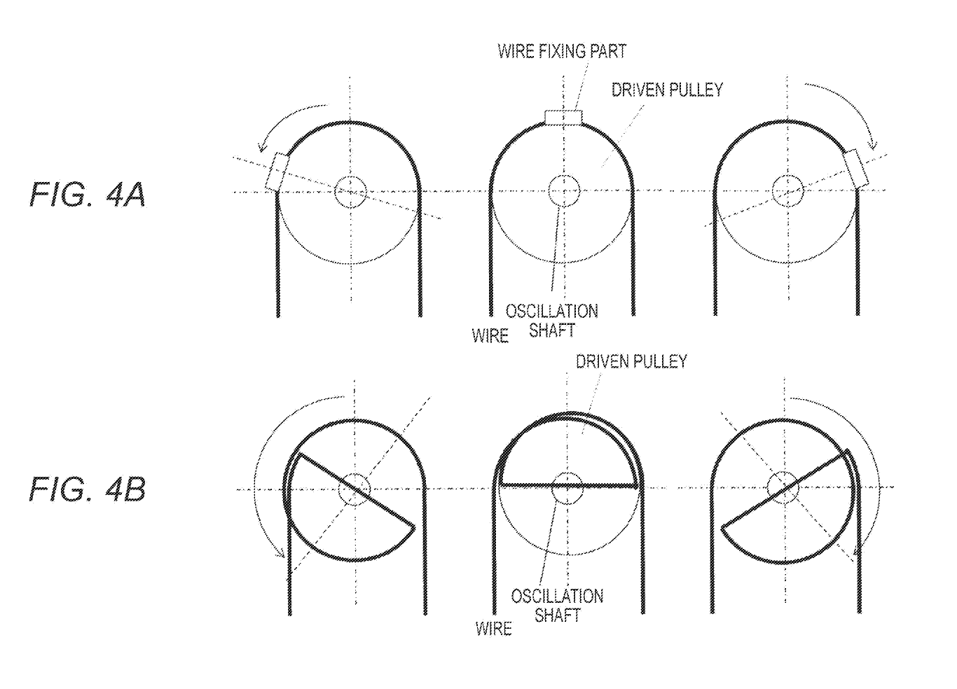

As illustrated in FIG. 4A, in a related art, a wire fixing part to fix a wire is generally provided in a groove of a driven pulley as a method of fixing a wire. In this case, since there is the wire fixing part, it has been difficult to rotate the driven pulley and a transducer unit for equal to or larger than 180°.

On the other hand, as illustrated in FIG. 4B, in an embodiment of the present invention, it is not necessary to provide a wire fixing part unlike the related art. Instead, a wire is arranged doubly. Thus, an oscillation angle of each of the driven pulley and the transducer unit can be equal to or larger than 180°.

Next, a detail of a method of sealing the holes 23a and 23b will be described with reference to FIG. 5. As illustrated in FIG. 5, holding members 80a and 80b which respectively hold the flexible printed circuits 70a and 70b are respectively inserted into the holes 23a and 23b. In this state, an adhesive is injected into the holes 23a and 23b and the holes 23a and 23b are sealed.

Accordingly, in a region in which a movement can be performed due to the oscillation of the transducer unit 50, the flexible printed circuits 70a and 70b are fixed to the transducer holding frame 20 without attachment of an adhesive.

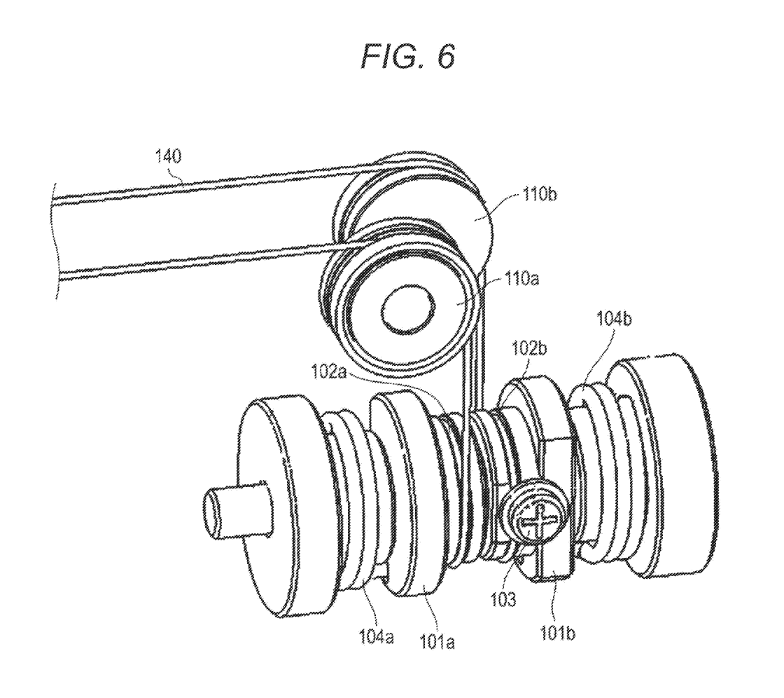

Next, a detail of the driving pulley unit 100 will be described with reference to FIG. 6. As illustrated in FIG. 6, in the driving pulleys 101a and 101b, wire grooves 102a and 102b to which the wire 140 is arranged are provided. Both end parts of the wire 140 are respectively fixed to the driving pulleys 101a and 101b by a screw lock in a wire end fixing part 103 while being arranged in the wire grooves 102a and 102b.

Further, to the driving pulley unit 100, coil springs 104a and 104b are attached. It is possible to apply tension to the wire 140 by biasing the coil springs 104a and 104b and it is possible to adjust the tension of the wire 140 by a degree of the biasing. Accordingly, it is possible to apply necessary tension to the wire 140 without providing a new tensioner which generates tension and to reduce a size and weight of the driving pulley unit. Further, accordingly, the wire 140 can oscillate the transducer unit 50 according to an amount of rotation of the output shaft 121 along with the driving of the motor 120.

Next, operation of the ultrasonic probe 1 will be described. First, an operator holds the grip part 11 and inserts the insertion part 12 into a body cavity.

Then, the operator operates an operation unit (not illustrated) of the diagnosis device main body and gives an instruction for forming an ultrasonic image. When the instruction is received, a control unit (not illustrated) of the diagnosis device main body oscillates and rotates the output shaft 121 (driving pulley unit 100) of the motor 120. Accordingly, a rotational movement of the driving pulley unit 100 is transmitted by the wire 140 to the driven pulley 40, whereby the transducer unit 50 can be oscillated and rotated around a rotational axis and the ultrasonic wave forming surface can be moved.

Further, the control unit of the diagnosis device main body transmits an electric signal to each transducer through the flexible printed circuits 70a and 70b. Along with the oscillation of the transducer unit 50, each transducer converts the received electric signal into an ultrasonic wave and emits the ultrasonic wave to an inner side of a body of a subject. Then, each transducer detects the ultrasonic wave reflected on the inner side of the body (reflection wave), converts the ultrasonic wave into an electric signal, and transmits the electric signal to the control unit of the diagnosis device main body through the flexible printed circuits 70a and 70b. Accordingly, the control unit can form a three-dimensional ultrasonic image and can display the ultrasonic image on a screen.

As described above, according to an embodiment of the present invention, since the transducer holding frame 20 and the casing 90 are connected by the pipes 130a and 130b and the wire 140 is put through the pipes 130a and 130b, only the inside of each of the pipes 130a and 130b needs to be filled with coupling liquid in the insertion part 12 of the housing 10. Thus, it is possible to reduce an amount of the coupling liquid and to reduce weight of the ultrasonic probe 1.

Further, according to an embodiment of the present invention, each of the metal pipes 130a and 130b has predetermined intensity. Thus, it is possible to form the insertion part 12 of the housing 10 from a light material such as plastic and to reduce weight of the ultrasonic probe 1.

Further, according to an embodiment of the present invention, the flexible printed circuits 70a and 70b are arranged outside of the pipes 130a and 130b. Thus, it is possible to reduce a part, which is in contact with coupling liquid, of each of the flexible printed circuits 70a and 70b and to prevent the flexible printed circuits 70a and 70b from being damaged due to a contact with the wire 140.

Further, according to an embodiment of the present invention, the two wire grooves 42 and 43 and the hole 44 to connect the two are formed in the driven pulley 40 and the wire 140 is doubly arranged in the driven pulley 40 in such a manner that the wire 140 arranged in the wire groove 42 is put through the hole 44 and is arranged in the wire groove 43. Thus, an oscillation angle of the transducer unit 50 can be increased compared to a related art.

Note that in the above-described embodiment, a case where there are two pipes has been described but the present invention is not limited thereto. There may be one pipe.

An embodiment of the present invention can be used for an ultrasonic probe which is inserted into a body cavity and which performs scanning with an ultrasonic transducer automatically.

Although the present invention has been described and illustrated in detail, it is clearly understood that the same is by way of illustrated and example only and is not to be taken by way of limitation, the scope of the present invention being interpreted by terms of the appended claims.

What is claimed is:

1. An ultrasonic probe comprising:
a transducer unit including a plurality of transducers configured to transmit/receive an ultrasonic wave;
a transducer holding frame which holds the transducer unit in an oscillatable manner;
an acoustic window which is formed from a material having an ultrasonic transmission property, provided in a front part of an ultrasonic wave transmission/reception surface of the transducer unit, and fixed to the transducer holding frame in a sealed manner, the acoustic window forming a first space including the transducer unit in an inner part;
a driving unit configured to generate drive force to oscillate the transducer unit;
a driving unit holding frame which holds the driving unit and forms a second space including an output shaft of the driving unit in an inner part;
a pipe including a hollow portion with which connects the first space and the second space;
a force transmission unit which is put through the hollow portion of the pipe and which is configured to oscillate the transducer unit by transmitting the drive force of the driving unit; and
a housing including, in an inner part, the transducer unit, the transducer holding frame, the acoustic window, the driving unit, the driving unit holding frame, the pipe, and the force transmission unit,
wherein the first space, the hollow portion of the pipe, and the second space are filled with coupling liquid.

2. The ultrasonic probe according to claim 1, further comprising a driven pulley coupled to the transducer unit, and
two driving pulleys which are housed in the inner part of the second space, fixed to the output shaft of the driving unit, and rotated by the drive force of the driving unit,
wherein the force transmission unit is a wire which is arranged in the driven pulley and the two driving pulleys and both ends of which are respectively fixed to the two driving pulleys.

3. The ultrasonic probe according to claim 2, wherein the two driving pulleys are arranged in such a manner that rotational axes thereof are orthogonal to a rotational axis of the driven pulley,
two intermediate pulleys which are housed in the inner part of the second space and which are arranged in such a manner that rotational axes thereof are orthogonal to the rotational axis of the driven pulley and the rotational axes of the two driving pulleys are further included, and
the wire is arranged in the two intermediate pulleys between the driven pulley and the two driving pulleys.

4. The ultrasonic probe according to claim 2, wherein two grooves and a coupling hole to connect the two grooves are formed in an outer peripheral part of the driven pulley, and
the wire is arranged doubly in the driven pulley in such a manner as to be arranged in one of the grooves, put through the coupling hole, and arranged in the other groove.

5. The ultrasonic probe according to claim 1, further comprising flexible printed circuits which are connected to the transducers and which are configured to perform transmission/reception of an electric signal to/from the transducers, wherein
a sealing hole to put the flexible printed circuits outside of the pipe is formed in the transducer holding frame.

6. The ultrasonic probe according to claim 5, wherein the sealing hole is sealed in a state in which a holding member holding the flexible printed circuits is inserted therein.
